(19) United States
(12) Patent Application Publication
(10) Pub. No.: US 2023/0223153 A1
(43) Pub. Date: Jul. 13, 2023

(54) PREDICTION OF QUALITY OF LIFE IN PATIENTS WITH TRAUMATIC BRAIN INJURY

(71) Applicants: Regents of the University of Minnesota, Minneapolis, MN (US); The Medical College of Wisconsin, Inc., Milwaukee, WI (US)

(72) Inventors: Christopher Tignanelli, Edina, MN (US); Benjamin Chen, Shoreview, MN (US); Rachel Morris, Fox Point, WI (US)

(21) Appl. No.: 18/152,487

(22) Filed: Jan. 10, 2023

Related U.S. Application Data
(60) Provisional application No. 63/266,612, filed on Jan. 10, 2022.

(51) Int. Cl. G16H 50/50; G16H 50/70

(52) U.S. Cl. CPC G16H 50/50; G16H 50/70

(57) ABSTRACT

A computer-based system that includes one or more computing devices configured to receive user input comprising a plurality of parameters indicating a condition of a trauma patient, the plurality of parameters comprising at least one patient reported outcome; apply the plurality of parameters to a predictive model trained to determine, based on the plurality of parameters, a quality of life designation for the patient; and transmit the quality of life designation to a user computing device for display on the user computing device.

This application claims the benefit of U.S. Provisional Patent Application No. 63/266,612, filed Jan. 10, 2022, the entire contents of which is incorporated herein by reference.

### TECHNICAL FIELD

The disclosure relates to medical computing systems, and in particular, computing systems configured to predict patient outcomes for patients with brain injury.

### BACKGROUND

Every year in the United States, more than 2.53 million patients visit the Emergency Department (ED) due to a traumatic brain injury (TBI). This has resulted in 13.5 million Americans with TBI-associated disability and/or reduced quality of life. Many of these patients face daily challenges secondary to TBI, and some never return to a full and productive life after injury.

### SUMMARY

In general, this disclosure describes computer-implemented systems and techniques for predicting a quality of life for a patient that has experienced traumatic brain injury (TBI). For example, a system may train a predictive model using medical record data and patient reported outcomes from a plurality of subjects. A system may then apply the trained predictive model to medical record data and patient reported outcomes for a subsequent patient in order to predict the quality of life for that patient.

In some examples, a computing system includes processing circuitry configured to: receive user input comprising a plurality of parameters indicating a condition of a trauma patient, the plurality of parameters comprising at least one patient reported outcome; apply the plurality of parameters to a predictive model trained to determine, based on the plurality of parameters, a quality of life designation for the patient; and transmit the quality of life designation to a user computing device for display on the user computing device.

In some examples, a method includes receiving, by processing circuitry, user input comprising a plurality of parameters indicating a condition of a trauma patient, the plurality of parameters comprising at least one patient reported outcome; applying, by the processing circuitry, the plurality of parameters to a predictive model trained to determine, based on the plurality of parameters, a quality of life designation for the patient; and transmitting, by the processing circuitry, the quality of life designation to a user computing device for display on the user computing device.

In some examples, a non-transitory computer-readable medium comprising instructions that, when executed, cause processing circuitry to receive user input comprising a plurality of parameters indicating a condition of a trauma patient, the plurality of parameters comprising at least one patient reported outcome; apply the plurality of parameters to a predictive model trained to determine, based on the plurality of parameters, a quality of life designation for the patient; and transmit the quality of life designation to a user computing device for display on the user computing device.

### BRIEF DESCRIPTION OF DRAWINGS

FIG. 1 is a flow chart of an example technique for generating a predictive model based on patient TBI data.

FIG. 2 is a conceptual block diagram illustrating an example computing system for determining quality of life for patients with traumatic brain injury.

FIG. 3 is a conceptual block diagram illustrating various example computing devices of the computing system of FIG. 2.

FIG. 4 is a flowchart illustrating an example operation in accordance with the present techniques.

### DETAILED DESCRIPTION

This disclosure describes a prediction model and techniques, systems, and devices that can utilize the prediction model to predict quality of life in trauma patients with traumatic brain injury (TBI). This quality of life may be predicted with reasonable accuracy at 3-month, 6-month, and 12-month timelines, for example, using easily available medical record data and patient reported outcomes.

As discussed above, many patients that have experienced TBI face daily challenges secondary to TBI, and some never return to a full and productive life after injury. Predicting which patients will have favorable quality of life outcomes is important to guide treatment decisions. For example, Older patients with TBI suffer worse health-related outcomes when compared with younger patients with similar injuries.

In-hospital mortality and post-discharge quality of life (QOL) are difficult but important to predict in the trauma population. Predictive models have focused on mortality outcomes rather than quality of life. QOL outcomes can be difficult to predict due to the lack of large multicenter center databanks. Validated predictive models, such as Corticosteroid Randomization after Significant Head Injury (CRASH) and international Mission for Prognosis and Clinical Trials in Traumatic Brain Injury (IMPACT), have solely focused on moderate and severe TBI. The lack of an accurate, widely-applicable and usable QOL prediction model represents a gap in the current knowledge of TBI outcomes.

Predictive tools can be helpful for providers and patients to avoid bias and provide additional objective information during shared decision-making conversations. Absence of prediction models may lead to potentially expensive and invasive treatments in the face of high morbidity and mortality or inappropriate withdrawal of life sustaining therapies. Predictive models for quality of life metrics have traditionally been limited by small studies lacking long-term follow-up, lack of TBI-specific QoL measures, and logistical challenges of collecting quality of life survey data.

Current prediction of QOL can be based solely on the patient's in-hospital anatomic and physiologic status. Progress has been made in predicting mortality in older adults. The Elderly Mortality After Trauma (EMAT) score was developed to predict in-hospital mortality. Patients often value quality of life over quantity at the end of their life. Yet, previous studies in adult TBI emphasized mortality over Patient Reported Outcomes (PROs). Patient reported outcomes measures (PROMs) assess the quality of life provided from the patient perspective, which is important in patients whose QOL may be especially affected by injury. Multiple health-related quality of life (HRQoL) instruments have been tested in TBI, including the Quality of Life After Brain Injury (QOLIBRI) overall scale, with good representation of TBI-specific QoL. However, no prediction models exist to predict QOLIBRI using large databanks. A significant limitation in this effort is the lack of large datasets, and those datasets that include post-discharge PROMs to develop predictive models. Such predictive models can then aid physician, patient, and family decision making regarding treatment in the hospital.

In some examples, a post-hospital discharge TBI-specific PROM could be predicted using variables readily available early in the hospitalization. As described herein, a prediction model, TBI-PRO, has been developed and validated that predicts quality of life in trauma patients with TBI with reasonable accuracy at 3-month, 6-month, and 12-month intervals using easily available medical record data and patient reported outcomes. In other words, a system may employ the predictive model to generate a score based on available medical record data and patient reported outcomes. This score may be useful as a tool in shared decision-making conversations with patients who have suffered a TBI and their families.

The following methods are one example for developing, validating, and executing a predictive model for determining quality of life for patients that have experienced TBI. These are non-limiting examples, as other methods and data may be used in other examples.

Methods and Data

The Transforming Research and Clinical Knowledge in Trauma Brain Injury (TRACK-TBI) (2014-2018) is a prospective multicenter study of level 1 trauma centers in the United States. The TRACK-TBI database includes patient, imaging, laboratory, and hospital data on traumatic brain injury patients, as well as clinical/PROs from 18 sites collected with a standardized data dictionary and utilizing the NIH-NINDS TBI Common Data Elements (TBI-CDEs). The institutional review board for each center in TRACK-TBI approved the study described herein and study staff obtained consent from all patient participants and/or their legally authorized representatives.

Participants

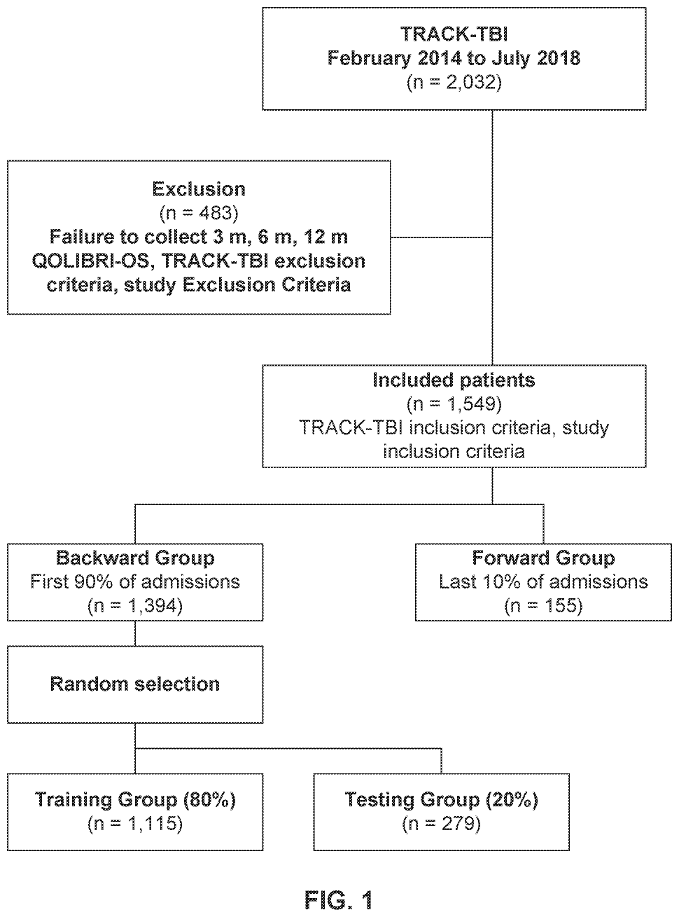

As shown in FIG. 1, patient-level data were obtained from TRACK-TBI for cases admitting between Feb. 26, 2014 through Jan. 19, 2018 for the “backward” training (n=1115) and testing (n=279) datasets, and Jan. 20, 2018 through Jul. 3, 2018 (last 10% of admitted) for the “forward” validation dataset (n=155). The “backward” dataset was divided into training and testing cohorts randomly.

The TRACK-TBI inclusion criteria were as follows: for the patient to present within 24 hours of injury, have an acute head CT scan performed as part of clinical care, and show or report evidence of alterations in consciousness or amnesia. Exclusions included being in custody, pregnant, nonsurvivable physical trauma, debilitating mental health disorders, neurologic disease, or non-English speaking; however, some sites recruited Spanish-speaking participants.

The criteria for this analysis included: traumatic brain injured defined as in the TRACK-TBI inclusion criteria, age ≥18 years, admitted to hospital (medical floor or ICU), completed QOLIBRI-OS or documented significant death/disability at 3 months, 6 months, or 12 months. The analysis-specific exclusion criteria were: TRACK-TBI exclusion criteria, no signs of life at initial evaluation (Emergency Department (ED) Systolic blood pressure (SBP)=0, pulse=0), discharge home from the ED, failure to complete 3 month-12-month QOLIBRI measurement due to loss to follow-up.

Procedure and Measures

The study sample was divided into a backward dataset for training (first 90% of admissions) and a forward dataset for validation (10% most recent admissions). The backward dataset group was subsequently randomly divided into an 80% training and a 20% testing dataset. Predictions derived from the training dataset were applied to the testing and forward validation datasets. Patients who ultimately died from their injuries were included in the model as mortality is unknown at the time the model is clinically applied.

The backward dataset was used to derive the traumatic brain injury-patient reported outcome (TBI-PRO) model. The model was ultimately validated temporally in the forward dataset. Temporal validation was performed to study the generalizability of the model's results across time. To address missing data, the mice package in R was utilized to conduct predictive mean matching multiple imputation, with 5 iterations and a total of 10 imputed datasets. The final imputed dataset was randomly selected. The dependent variable, 3-month QOLIBRI, was not imputed. Separate datasets were also created for 6-month and 12-month QOLIBRI scores. The Area Under the Receiver Operating Characteristic (AUROC) curve was reported for the forward dataset.

The TBI-PRO was developed based on data presumed to be available at 24 hours after admission. The following candidate variables (provided for each category of variables) can be used for model derivation in some examples. Demographics: Age at injury, Sex, Race, Body Mass Index, Time from injury to admission, and Insurance. Anatomic/Physiologic status: Systolic Blood Pressure (lowest on day 0-3), Heart Rate (highest on day 0-3), Temperature (highest on day 0-3), Respiratory Rate (highest on day 0-3), Oxygen Saturation (lowest on day 0-3), Glasgow Coma Scale (highest on day 0-3), Number of reactive pupils (lowest on day 0-3), and Major extracranial injury. Past Medical History: Cerebrovascular Accident, Hypertension, Chronic Obstructive Pulmonary Disease, Cirrhosis, Renal insufficiency, Cancer, Heart disease, Neurologic Disease, Psychiatric Disease, Developmental Disease, Eye, Ear, Nost & Throat Disease, Cardiovascular Disease, Pulmonary Disease, Gastrointestinal Disease, Hepatic Disease, Renal Disease, Endocrine Disease, Hematologic Disease, Musculoskeletal Disease, Spinal Disease, Pre-injury steroid use, Pre-injury anticoagulant use, Alcohol use (AUDIT score), Current smoker, and Written DNR during hospital stay. Laboratory/Imaging: White Blood Cells (highest/lowest on day 0-3), Hemoglobin (highest/lowest on day 0-3), Hematocrit (highest/lowest on day 0-3), Platelets (highest/lowest on day 0-3), International Normalized Ratio (highest/lowest on day 0-3), Prothrombin Time (highest on day 0-3), Partial Thromboplastin Time (highest on day 0-3), Sodium (highest/lowest on day 0-3), Potassium (highest/lowest on day 0-3), Bicarbonate (highest/lowest on day 0-3), Creatinine (highest on day 0-3), Urea (highest on day 0-3), Initial Head CT: Subdural Hematoma, Initial Head CT: Intracranial Hematoma, Initial Head CT: Midline shift, Initial Head CT: Contusions, Initial Head CT: Epidural Hematoma, and Initial Head CT: Edema. Interventions: Intracranial Pressure (highest on day 0), Intracranial Pressure (highest on day 2-3), ICP monitor used, Craniectomy, Number of neurosurgical procedures on day 0, Number of neurosurgical procedures on day 2-3, Number of non-neurosurgical procedures on day 0, Number of non-neurosurgical procedures on day 2-3, Tracheostomy, Percutaneous Gastrostomy procedure, and Need for dialysis.

The above candidate variables can be initially chosen based on available data from TRACK-TBI that published evidence suggests would be likely to influence quality of life outcomes. The TRACK-TBI data dictionary's definition and data fields were used to select variables including demographics (pre-injury living situation, study site, body mass index, insurance status, age, race, sex), and comorbidities (cardiovascular, endocrine, gastrointestinal, hematologic, musculoskeletal, hepatic, spinal, neurologic, pulmonary, renal, developmental, steroid use, smoking status, pre-injury anticoagulant use and alcohol use). Laboratory values collected included white blood cell count, hemoglobin, hematocrit, platelets, sodium, international normalized ratio (INR), prothrombin time (PT), partial thromboplastin time (PTT), creatinine, urea, potassium, and bicarbonate at admission. The most aberrant physiologic parameters as high and low values (systolic blood pressure (SBP), pulse, temperature, Glasgow Coma Scale [GCS] and respiratory rate). SBP, heart rate, GCS, respiratory rate, temperature, oxygen saturation, and laboratory values were collected at 24 hours after admission. Comorbidities with a prevalence of less than <1% were not included in the model. The data elements for neurologic exam were number of reactive pupils and GCS on hospital day 1. On admission CT, the following variables were included: subdural hematoma, midline shift, intracranial hemorrhage, cerebral edema, extradural hemorrhage, and contusion. All clinical data were entered into electronic Case Report Forms (eCRFs) and managed by the QuesGen data management platform. Select data were manually abstracted for analysis. For treatment interventions, ICP monitor, craniectomy, need for dialysis, tracheostomy, number of neurologic procedures (up to 24 hours after admission), and percutaneous endoscopic gastrostomy were included.

The Quality of Life after Brain Injury overall scale (QOLIBRI-OS) was used to assess patient-reported TBI-related QoL. The QOLIBRI-OS is a 6-item summary measure used to provide an index of quality of life after TBI. It correlates well with the full QOLIBRI scale. QOLIBRI-OS scores were collected at 3, 6, and 12 months after injury. The QOLIBRI-OS score (range 0-100) was dichotomized based on previously established cutoffs in the literature; 52% or less represents non-favorable quality of life. For patients unable to complete the QOLIBRI-OS questionnaire due to death or disability, the QOLIBRI-OS was coded as unfavorable. Deaths were included in the sample because discharge disposition would be unknown at the time of clinical use and to avoid exclusion of patients with the most severe TBI.

Outcomes

The primary outcome of interest was favorable vs non-favorable QoL at 3, 6, and 12 months after injury (based on dichotomized QOLIBRI-OS score, with non-favorable QoL inferred for individuals with incomplete QOLIBRI-OS due to death or significant disability). In other examples, the outcome of interest may be a larger range of scores (e.g., a range of 1 through 5 or 1 through 10 with the larger numbers indicating a more favorable outcome) as opposed to a binary favorable vs. non-favorable approach.

Statistical Methods

Logistic regression of QOLIBRI-OS was performed using data available within the first 24 hours after admission (TBI-PRO) (demographics, injuries, CT scan findings, laboratory values, physiologic parameters and co-morbidities). Laboratory values for the model were derived from admission laboratory values. Site was included in the model as a control variable. Ultimately, a p<0.05 cut-off for statistical significance was utilized for variable selection. Therefore, variables with a p<0.05 were included in a reduced logistic regression model to identify independent predictors of favorable outcome.

Student's t-tests were performed to explore differences between the two groups for continuous variables and chi-squared tests of independence for categorical variables. Statistical significance was defined as p<0.05 and all tests were two-tailed. Data are expressed as the mean±standard deviation (S.D.) for continuous descriptive variables and percentages for categorical variables. All data analyses were performed in R (R Core Team, 2020) and IBM SPSS Statistics Version 24.0 (Armonk, N.Y.: IBM Corp.).

Results

Of 2032 admissions, 1,549 participants met inclusion criteria; 1,394 in the backward group (1,115 in the training group, 279 in the testing group) and 155 in the forward group (FIG. 1). The most common reason for exclusion was missing the 3-month QOLIBRI-OS score. Fifty-six percent of cases had a favorable 3-month QOLIBRI-OS score. Patients in the training cohort were older, less likely to be white, and had a high GCS, compared with those in the validation dataset (Table 1).

TABLE 1
Patient characteristics in the Training and Prospective sets.

| | Training Set | | Prospective Set | | p- |
|---|---|---|---|---|---|
| Variable | N = 1115 (%) | | N = 155 (%) | | value |
| Age, years, | 38.5 | (26.5, 56.5) | 46.5 | (28.5, 60.5) | 0.023 |
| median (IQR) | | | | | |
| Female gender (%) | 331 | (29.7) | 44 | (28.4) | 0.706 |
| White race (%) | 882 | (79.1) | 127 | (81.9) | 0.004 |
| ISS, median (IQR) | 14.0 | (6.0, 21.0) | 13.0 | (5.0, 26.0) | 0.430 |
| Private Insurance (%) | 882 | (79.1) | 85 | (73.5) | 0.276 |
| Physiologic | | | | | |
| BMI, Kg/m2, | 26.5 | (23.4, 30.0) | 26.8 | (23.7, 30.1) | 0.747 |
| median (IQR) | | | | | |
| SBP, mmHg, mean | 114.3 | (±14.4) | 112.5 | (±12.9) | 0.124 |
| (SD) (lowest on | | | | | |
| day 0), | | | | | |
| HR, bpm, mean (SD) | 91.2 | (±16.7) | 93.1 | (±18.8) | 0.197 |
| (highest on day 0) | | | | | |
| GCS, mean (SD) | 12.3 | (±4.3) | 11.1 | (±4.9) | 0.003 |
| (highest on day 0) | | | | | |
| Past Medical | | | | | |
| History (%) | | | | | |
| Hypertension | 199 | (18) | 30 | (19.4) | 0.688 |
| COPD | 14 | (1.3) | 3 | (1.9) | 0.490 |
| Cancer | 46 | (4.1) | 5 | (3.2) | 0.593 |
| Neurologic | 156 | (14) | 20 | (12.9) | 0.713 |
| Psychiatric | 261 | (23.4) | 35 | (22.6) | 0.819 |
| Developmental | 87 | (7.8) | 7 | (4.5) | 0.143 |
| Eye, Ear, | 159 | (14.3) | 7 | (4.5) | 0.014 |
| Nose & Throat | | | | | |
| Cardiovascular | 256 | (23) | 36 | (23.2) | 0.961 |
| Pulmonary | 137 | (12.3) | 18 | (11.6) | 0.810 |
| Gastrointestinal | 167 | (15) | 20 | (12.9) | 0.495 |
| Hepatic | 23 | (2.1) | 2 | (1.3) | 0.517 |
| Endocrine | 211 | (18.9) | 39 | (25.2) | 0.067 |
| Hematologic | 39 | (3.5) | 5 | (3.2) | 0.862 |
| Musculoskeletal | 216 | (19.4) | 24 | (15.5) | 0.247 |
| Spinal | 52 | (4.7) | 5 | (3.2) | 0.418 |
| Pre-injury | 130 | (11.7) | 21 | (13.5) | 0.518 |
| Anticoagulant Use | | | | | |

Abbreviations: IQR, Interquartile range; ISS, Injury Severity Score; BMI, Body Mass Index; Kg/m2, Kilograms over square meters; SBP, Systolic Blood Pressure; HR, Heart Rate; bpm, beats per minute; GCS, Glasgow Coma Scale; COPD, Chronic Obstructive Pulmonary Disease.

Patients with favorable QOLIBRI-OS scores at 3 months were more likely to be younger, white race, high GCS and lower total ISS (Table 2). All physiologic parameters on hospital day 0 of admission were significant. Pre-injury use of anticoagulation was not significantly different between groups (p=0.08) (Table 2). White race was associated with a favorable QOLIBRI-OS score.

TABLE 2
Demographics of patients with favorable
and unfavorable QOLIBRI outcomes.

| | QOLIBRI | | QOLIBRI | | |
|---|---|---|---|---|---|
| | Favorable | | Unfavorable | | |
| | Outcome | | Outcome | | p- |
| Variables | N = 873 (%) | | N = 676 (%) | | value |
| Age, years, | 36.5 | (25.5, 54.5) | 45.5 | (28.5, 58.5) | 0.001 |
| median (IQR) | | | | | |
| Female gender (%) | 240 | (27.5) | 213 | (31.5) | 0.068 |
| White race (%) | 702 | (80.4) | 522 | (77.2) | 0.001 |
| ISS, median (IQR) | 11.0 | (5.0, 17.0) | 17.0 | (9.0, 26.0) | 0.001 |
| Private | 733 | (84.0) | 486 | (71.9) | 0.001 |
| Insurance (%) | | | | | |
| Physiologic | | | | | |
| BMI, Kg/m2, | 26.3 | (23.4, 29.9) | 26.8 | (23.4, 30.3) | 0.076 |
| median (IQR) | | | | | |
| SBP, mmHg, mean | 114.8 | (±13.3) | 113 | (±15.2) | 0.014 |
| (SD) (lowest on | | | | | |
| day 0) | | | | | |
| Heart Rate, bpm, | 90 | (±15.8) | 94 | (±18.4) | 0.001 |
| mean (SD) | | | | | |
| (highest on day 0) | | | | | |
| GCS, mean (SD) | 13.4 | (±3.3) | 10.6 | (±5) | 0.001 |
| (highest on day 0) | | | | | |
| Pre-injury | 97 | (11.1) | 95 | (14.1) | 0.081 |
| Anticoagulant Use | | | | | |
| Past Medical | | | | | |
| History (%) | | | | | |
| Hypertension | 122 | (14) | 155 | (22.9) | 0.001 |
| COPD | 3 | (0.30) | 16 | (2.4) | 0.001 |
| Cancer | 28 | (3.2) | 33 | (4.9) | 0.093 |
| Neurologic | 90 | (10.3) | 118 | (17.5) | 0.001 |
| Psychiatric | 160 | (18.3) | 204 | (30.2) | 0.001 |
| Developmental | 69 | (7.9) | 41 | (6.1) | 0.16 |
| Eye, Ear, | 121 | (13.9) | 91 | (13.5) | 0.821 |
| Nose & Throat | | | | | |
| Cardiovascular | 172 | (19.7) | 183 | (27.1) | 0.001 |
| Pulmonary | 88 | (10.1) | 97 | (14.3) | 0.01 |
| Gastrointestinal | 107 | (12.3) | 122 | (18) | 0.001 |
| Hepatic | 8 | (0.9) | 28 | (4.1) | 0.001 |
| Endocrine | 137 | (15.7) | 159 | (23.5) | 0.001 |
| Hematologic | 30 | (3.4) | 32 | (4.7) | 0.196 |
| Musculoskeletal | 159 | (18.2) | 139 | (20.6) | 0.245 |
| Spinal | 31 | (3.6) | 37 | (5.5) | 0.067 |

Abbreviations: IQR, interquartile range; ISS, Injury Severity Score; BMI, Body Mass Index; Kg/m2, Kilograms over square meters; SBP, Systolic Blood Pressure; bpm, beats per minute; HR, Heart Rate; GCS, Glasgow Coma Scale; COPD, Chronic Obstructive Pulmonary Disease; Cancer: Leukemia, lymphoma, breast, prostate, lung and kidney cancer; Neurologic: Spinal cord injury, vertebral injury, cerebrovascular anomaly, tumor, Cerebrovascular accident, transient ischemic attack, seizures, epilepsy, migraine, previous traumatic brain injury; Psychiatric: Anxiety, depression, sleep disorders, schizophrenia; Developmental: learning disabilities, attention deficit/hyperactivity disorder, developmental delay; Eye, Ear, Nose & Throat: Sinusitis, vision abnormality, hearing deficit; Cardiovascular: congenital heart disease, arrhythmias, ischemic heart disease, valvular disease, hypertension, thromboembolic disease, peripheral vascular disease; Pulmonary: COPD, asthma, pneumonia, tuberculosis; Gastrointestinal: Gastroesophageal reflux disease, gastrointestinal bleed, inflammatory bowel disease; Hepatic: hepatic insufficiency, failure, hepatitis, cirrhosis; Endocrine: thyroid disorders, diabetes mellitus type I and II; Hematologic: Anemia, HIV, AIDS, coagulopathy; Musculoskeletal: Arthritis, pressure ulcers.

Separate models were constructed using the same statistical methods to predict longer term outcomes (6-month and 12-month QOLIBRI-OS scores).

Model Derivation and Performance

Multiple logistic regression analysis identified 22 independent associations with favorable QOLIBRI-OS in the 3-month model (Table 3).

TABLE 3
TBI-PRO model variables with weight for 3-month QOLIBRI.

| | | |
|---|---|---|
| | Coefficient | p- |
| Variable | (SE) | value |

| Time from injury | −0.044 | (0.021) | <0.05 |
|---|---|---|---|
| to admission | | | |
| Age, years | | | |
| 45-54 | −0.643 | (0.175) | <0.01 |
| 55-64 | −0.937 | (0.191) | <0.01 |
| 65-74 | −0.285 | (0.274) | >0.1 |
| 75 or more | −0.656 | (0.357) | <0.1 |
| Race | | | |
| Black | −0.588 | (0.169) | <0.01 |
| Other Race | 0.607 | (0.287) | <0.05 |
| Physiologic | | | |
| SBP, mmHg, | 0.010 | (0.004) | <0.05 |
| (lowest on day 0) | | | |
| RR (highest on day 0) | −0.040 | (0.015) | <0.01 |
| SpO2 (lowest on day 0) | −0.045 | (0.020) | <0.05 |
| GCS (highest on day 0) | 0.079 | (0.018) | <0.01 |
| Number of reactive pupils | 0.434 | (0.131) | <0.01 |
| (lowest on day 0) | | | |
| Laboratory and | | | |
| Imaging Studies | | | |
| PTT (highest on day 0) | −0.016 | (0.010) | >0.1 |
| Potassium (lowest on day 0) | 0.430 | (0.134) | <0.01 |
| Bicarbonate (highest on day 0) | 0.012 | (0.019) | >0.1 |
| Initial CT: midline shift | −0.064 | (0.028) | <0.05 |
| Initial CT: edema | −0.599 | (0.202) | <0.01 |
| Past Medical | | | |
| History | | | |
| Hypertension | −0.978 | (0.309) | <0.01 |
| COPD | −1.178 | (0.699) | <0.1 |
| Neurologic | −0.482 | (0.180) | <0.01 |
| Psychiatric | −0.688 | (0.143) | <0.01 |
| Cardiovascular | 0.665 | (0.289) | <0.05 |
| Hepatic | −1.145 | (0.476) | <0.05 |
| Spinal | −0.462 | (0.285) | >0.1 |
| Smoker | −0.368 | (0.134) | <0.01 |
| Insurance | | | |
| Medicaid or similar | −0.831 | (0.193) | <0.01 |
| Medicare or similar | −0.265 | (0.273) | >0.1 |
| Constant | 2.110 | (2.303) | >0.1 |

Abbreviations: SBP, Systolic Blood Pressure; RR, Respiratory Rate; SpO2, Saturation Peripheral Oxygen; GCS, Glasgow Coma Scale; PTT, Partial Thromboplastin Time; CT, Computerized Tomography.

These included 1 time variable (time from injury to admission), 3 demographic, 5 physiologic, 8 comorbidities, and 5 laboratory or imaging variables. The 6-month model included 17 independent predictors (Table 4) and the 12-month model included 21 independent predictors (Table 5).

TABLE 4
TBI-PRO model variables with weight for 6-month QOLIBRI.

| | | |
|---|---|---|
| | Coefficient | p- |
| Variable | (SE) | value |

| Time from injury | −0.044 | (0.021) | <0.05 |
|---|---|---|---|
| to admission | | | |
| Age, years | | | |
| 45-54 | −0.496 | (0.180) | <0.01 |
| 55-64 | −0.822 | (0.184) | <0.01 |
| 65-74 | −0.275 | (0.280) | >0.1 |
| 75 or more | −0.988 | (0.357) | <0.01 |
| Race | | | |
| Black | −0.954 | (0.176) | <0.01 |
| Other Race | 0.537 | (0.303) | <0.1 |
| Physiologic | | | |
| SBP, mmHg, | 0.011 | (0.004) | <0.05 |
| (lowest on day 0) | | | |
| RR (highest on day 0) | −0.041 | (0.015) | <0.01 |
| GCS (highest on day 0) | 0.072 | (0.018) | <0.01 |
| Number of reactive pupils | 0.199 | (0.134) | >0.1 |
| (lowest on day 0) | | | |
| Major Extracranial Injury | −0.238 | (0.145) | >0.1 |
| Laboratory and | | | |
| Imaging Studies | | | |
| PTT (highest on day 0) | −0.020 | (0.010) | <0.05 |
| Initial CT: Midline shift | −0.098 | (0.027) | <0.01 |
| Initial CT: Edema | −0.640 | (0.208) | <0.01 |
| Initial CT: Epidural Hematoma | 0.356 | (0.223) | >0.1 |
| Past Medical History | | | |
| COPD | −1.938 | (0.788) | <0.05 |
| Psychiatric | −1.093 | (0.147) | <0.01 |
| Hepatic | −1.004 | (0.451) | <0.05 |
| Smoker | −0.601 | (0.143) | <0.01 |
| Insurance | | | |
| Medicaid or similar | −0.797 | (0.200) | <0.01 |
| Medicare or similar | −0.391 | (0.290) | >0.1 |
| Constant | 0.301 | (0.761) | |

Abbreviations: SBP, Systolic Blood Pressure; RR, Respiratory Rate; GCS, Glasgow Coma Scale; PTT, Partial Thromboplastin Time; CT, Computerized Tomography; COPD, Chronic Obstructive Pulmonary Disease

TABLE 5
TBI-PRO model variables with weight for 12-month QOLIBRI.

| | | |
|---|---|---|
| | Coefficient | p- |
| Variable | (SE) | value |

| Time from injury | −0.040 | (0.025) | >0.1 |
|---|---|---|---|
| to admission | | | |
| Age, years | | | |
| 45-54 | −0.289 | (0.195) | >0.1 |
| 55-64 | −0.763 | (0.206) | <0.01 |
| 65-74 | −0.402 | (0.300) | >0.1 |
| 75 or more | −0.577 | (0.386) | >0.1 |
| Race | | | |
| Black | −0.560 | (0.184) | <0.01 |
| Other race | 0.660 | (0.315) | <0.05 |
| Physiologic | | | |
| SBP (lowest | 0.008 | (0.005) | <0.1 |
| on day 0) | | | |
| Number of reactive pupils | 0.561 | (0.126) | <0.01 |
| (lowest on day 0) | | | |
| Laboratory and | | | |
| Imaging studies | | | |
| Sodium (highest on day 0) | −0.030 | (0.016) | <0.1 |
| Potassium (lowest on day 0) | 0.364 | (0.147) | <0.05 |
| Creatinine (highest on day 0) | −0.412 | (0.195) | <0.05 |
| Urea (highest on day 0) | 0.027 | (0.011) | <0.05 |
| Hematocrit (lowest on day 0) | 0.00002 | (0.012) | >0.1 |
| Platelets (highest on day 0) | −0.002 | (0.001) | <0.05 |
| PTT (highest on day 0) | −0.024 | (0.010) | <0.05 |
| Initial CT: Edema | −0.844 | (0.200) | <0.01 |
| Past Medical | | | |
| History | | | |
| Hypertension | −0.663 | (0.195) | <0.01 |
| Neurologic | −0.625 | (0.161) | <0.01 |
| Psychiatric | −0.692 | (0.153) | <0.01 |
| Gastroenterological | −0.502 | (0.181) | <0.01 |
| Endocrine | −0.474 | (0.187) | <0.05 |
| Smoker | −0.610 | (0.146) | <0.01 |
| Pre-injury | 0.240 | (0.224) | >0.1 |
| anticoagulant use | | | |
| Insurance | | | |
| Medicaid or similar | −0.842 | (0.212) | <0.01 |
| Medicare or similar | −0.255 | (0.288) | >0.1 |
| Constant | 3.989 | (2.688) | |

Abbreviations: SBP, Systolic Blood Pressure; PTT, Partial Thromboplastin Time; CT, Computerized Tomography.

Discrimination was assessed in the forward validation set: The 3-month model had an AUROC of 0.81 (95% CI 0.74-0.88) (FIG. 1). The model was tested at different thresholds (cutpoints for a positive test) and demonstrated varying sensitivity, specificity, positive predictive value and negative predictive value (Table 6). Characteristics of patients for whom the model correctly and incorrectly predicted favorable QOLIBRI-OS score is included in Table 7.

TABLE 6
Sensitivity and Specificity Analysis for 3-month Model.

| | | | Positive | Negative |
|---|---|---|---|---|
| Threshold | Sensitivity | Specificity | Predictive Value | Predictive Value |
| 0.06 | 0.99 | 0.08 | 0.58 | 0.98 |
| 0.91 | 0.01 | 0.99 | 0.91 | 0.44 |
| 0.60 | 0.70 | 0.70 | 0.75 | 0.64 |

TABLE 7
Correct versus Incorrect Model Predictions.

| | Correct | | Incorrect | | p- |
|---|---|---|---|---|---|
| Variable | N = 1088 (%) | | N = 461 (%) | | value |
| Female gender (%) | 308 | (28.3) | 145 | (31.5) | 0.214 |
| Age, years, | 38.5 | (26.5, 55.5) | 43.5 | (27.5, 58.0) | 0.042 |
| median (IQR) | | | | | |
| SS, median (IQR) | 14.0 | (8.0, 22.0) | 11.0 | (5.0, 18.0) | 0.001 |
| Private Insurance (%) | 864 | (79.4) | 355 | (77) | 0.071 |
| Physiologic | | | | | |
| BMI, Kg/m2, | 26.2 | (23.2, 29.7) | 27.0 | (24.0, 31.0) | 0.003 |
| median (IQR) | | | | | |
| SBP, mmHg, mean | 113.9 | (±14.5) | 114.3 | (±13.5) | 0.569 |
| (SD) (lowest | | | | | |
| on day 0) | | | | | |
| Heart Rate, bpm, | 91.7 | (±17.7) | 90.9 | (±15.5) | 0.375 |
| mean (SD) | | | | | |
| (highest on day 0) | | | | | |
| GCS, mean (SD) | 12.0 | (±4.5) | 12.7 | (±3.9) | 0.002 |
| (highest on day 0) | | | | | |
| Pre-injury | 126 | (11.6) | 66 | (14.3) | 0.135 |
| Anticoagulant Use | | | | | |
| Past Medical | | | | | |
| History (%) | | | | | |
| Hypertension | 184 | (16.9) | 93 | (20.2) | 0.126 |
| COPD | 16 | (1.5) | 3 | (0.7) | 0.180 |
| Cancer | 44 | (4) | 18 | (3.9) | 0.965 |
| Neurologic | 138 | (12.7) | 70 | (15.2) | 0.187 |
| Psychiatric | 254 | (23.3) | 111 | (24.1) | 0.726 |
| Developmental | 83 | (7.6) | 27 | (5.9) | 0.214 |
| Eye, Ear, | 151 | (13.9) | 61 | (13.2) | 0.735 |
| Nose & Throat | | | | | |
| Cardiovascular | 242 | (22.2) | 114 | (24.7) | 0.270 |
| Pulmonary | 123 | (11.3) | 61 | (13.4) | 0.234 |
| Gastrointestinal | 154 | (14.2) | 74 | (16.1) | 0.360 |
| Hepatic | 29 | (2.7) | 7 | (1.5) | 0.171 |
| Endocrine | 199 | (18.3) | 97 | (21.0) | 0.208 |
| Hematologic | 45 | (4.1) | 17 | (3.7) | 0.681 |
| Musculoskeletal | 207 | (19) | 91 | (19.7) | 0.744 |
| Spinal | 44 | (4) | 25 | (5.4) | 0.196 |

Abbreviations: IQR, interquartile range; ISS, Injury Severity Score; BMI, Body Mass Index; Kg/m2, Kilograms over square meters; SBP, Systolic Blood Pressure; bpm, beats per minute; HR, Heart Rate; GCS, Glasgow Coma Scale; COPD, Chronic Obstructive Pulmonary Disease.

The AUROCs for the 6-month and 12-month QOLIBRI-OS models were 0.79 (95% CI: 0.71-0.86) and 0.76 (95% CI: 0.68-0.84), respectively.

Subgroup Analysis

Group analysis was performed to assess model performance on underrepresented groups. It is unclear if the model performed less well in participants of Black race, AUROC 0.71 (CI 0.65-0.78) compared with White race AUROC 0.76 (95%: CI 0.75-0.80), with a wide confidence interval for the Black American AUROC. The model performs similarly by sex (males; AUROC 0.78 [95%: CI 0.76-0.81]; females; AUROC 0.75 [95%: CI 0.70-0.79]).

Discussion

The prospective multicenter TRACK-TBI dataset was used to create a predictive model (TBI-PRO) for favorable brain injury-related QoL at 3, 6, and 12 months using variables readily available in the hospital at 24 hours after injury. This predictive model predicts the QOLIBRI-OS patient-reported outcome measure with good discrimination of 0.81. In the specific example discussed herein, the performance of the model was not ascertainable for Black race due to underrepresentation of this population in the analytic cohort. However, these limitations may be removed in other example predictive models by including a broader representation of patients in the data set used to train the predictive model.

The lack of a validated TBI QOL model may represent a quality and knowledge gap which can leave patients vulnerable to non-beneficial interventions or inappropriate withdrawal of life-sustaining therapies. Designing models such as the predictive models described herein that can aid clinicians in helping patients and their loved ones understand the likelihood of favorable TBI-related QoL outcomes and what interventions lead to a favorable post-injury QoL is clinically relevant and important. For example, the system may also determine treatment plans based on the QoL determination for a particular patient. In some examples, the determination of a treatment plan may include excluding one or more treatment options from the treatment plan and/or including one or more specific treatment options for the treatment plan. Medicine is recognizing the importance of the perspective of the patient in healthcare and more research is needed to understand the importance of the interdependence of health needs, satisfaction, and quality of life.

The TBI-PRO model described herein has advantages, such as the advantage of training based on a multicenter, large dataset containing long-term PROMs. Discrimination ranges (0.70-0.82) were adequate when compared with previous models predicting QoL, as compared with those for predicting mortality. Although statistically significant, these differences may not be clinically meaningful. For example, patients with a GCS of 11 and 13 are likely to undergo similar treatments. A favorable QOLIBRI-OS score occurred in 50.3% of patients in the forward validation dataset.

It is unclear if the example model described here performed less well in participants of Black race, AUROC 0.71 (CI 0.65-0.78) compared with White race AUROC 0.76 (95%: CI 0.75-0.80), with a wide confidence interval for the Black American AUROC. It is unclear based on the data available in TRACK-TBI if specific sub-groups of patients, including older adults and certain races, are at above average risk of unfavorable outcomes. The post-trauma recovery of older patients may be prolonged and not reach the patient's expected goals. Data suggest that socioeconomic disparities between racial groups could further affect quality of life, as more socioeconomically advantaged individuals have better access to care, insurance coverage, and financial stability during times of recovery. From a statistical standpoint, race confounds outcomes and is significantly associated with unfavorable outcomes. Therefore, race was not excluded from the models.

Healthcare providers have difficulty finding a balance between beneficence and non-maleficence in their current practice as they lack reliable tools to assess post-discharge quality of life. There may be a need to develop a model to assess patient-reported outcomes in TBI patients. Historically, PROs were rarely linked to the patient's inpatient medical record, limiting the ability of research to correlate outcomes to inpatient variables.

Emphasis on quality of life after brain injury has yielded several prognostic models. The International Mission on Prognosis and Analysis of Clinical Trials in TBI (IMPACT) and the Corticosteroid Randomization After Severe Head Injury (CRASH) models predict 14-day mortality and unfavorable outcomes (death, vegetative state, severe disability as defined by Glasgow Outcome Scale) at 6 months after moderate to severe TBI, with AUROC curves ranging from 0.77 to 0.80, similar to TBI-PRO. In another study, the CRASH model had poor calibration for the outcome Glasgow Outcome Scale Extended (GOSE) and Persistent Post Concussive Symptoms (PPCS). The GOSE is a global scale for functional outcome that rates patient status into one of 8 categories: Dead, Vegetative State, Lower and Upper Severe Disability, Lower and Upper Moderate Disability, and Lower or Upper Good Recovery. In addition, the CRASH model overestimates the percentage of patients with poor outcomes. For PPCS, the models including 2-week post-injury symptoms had the best discriminative results. In a small study of 116 patients, investigators developed a long-term quality of life prediction model based on early hospital variables to predict unfavorable vs unfavorable QOLIBRI score 4 years after traumatic brain injury. This model had a moderate AUROC of 0.76. In this model, low GCS and preinjury working situations (other than employed or retired) were associated with non-favorable quality of life. Due to low patient volume, additional variables were unable to be analyzed necessitating a larger multicenter databank with more variables. The benefit of the example study discussed here is the utilization of a large prospective dataset to predict a long-term TBI-specific QoL metric, QOLIBRI-OS, at 3 month, 6 month, and 12 month post injury. The system and/or clinician may utilize these QoL metrics in order to determine a treatment plan. Such a determination may include excluding one or more treatment options and/or including one or more treatment options.

Other example studies and models may be adjusted in various ways. For example, QOLIBRI-OS was dichotomized, which may overlook non-linear effects and lead to a loss in power and effect size. However, clinicians, patients, and families may be more likely to desire a “blanket” prediction of “good” vs “bad” quality of life rather than a specific QOLIBRI score. Frailty and palliative care consultation were not included as these measures were not collected in TRACK-TBI, but these various could be utilized in developing other example predictive models. In other examples, example variables may include prior code status and frailty index because they may correlate with patient outcomes. Although the predictive model TBI-PRO described herein was tested over the time frame of 3, 6, and 12 months, the predictive model may still perform well predicting quality of life other shorter and longer timelines (e.g., longer than one year). Predictive models based on the variables described herein may have different accuracies in determining quality of life for patients with different groups of patients. For example, as discussed above, the example predictive model may be more accurate for patients of the white race than patients of the black race. In some examples, additional testing and/or adjustments to variables or weighting may be performed for patients of different races and/or other characteristics. For example, predictive model TBI-PRO may benefit from additional validation or development when applied to patients of certain underrepresented groups in the sample data, such as patients of the underrepresented minority black.

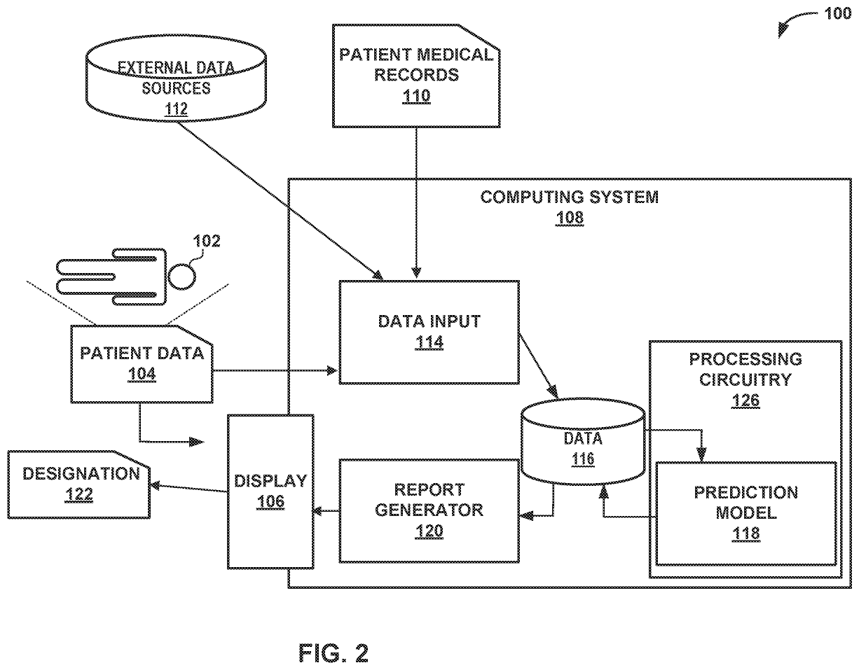

FIG. 2 is a block diagram illustrating an example quality of life (QOL) determination system 100 for determining an QOL designation 122 for a trauma patient 102. In general, QOL determination system 100 incorporates criteria (e.g., patient data 104) available to generate the model, such as hospitalization data (e.g., the patient medical record) and patient reported outcomes measures (PROMs).

More specifically, system 100, as described herein, is a system that can determine or predict a quality of life for a patient that experienced traumatic brain injury using information such as medical record information and patient reported outcomes. This information may be available during hospitalization. In this way, system 100 can more-accurately predict the QOL of trauma patient 102 which can be used to direct subsequent care for the patient 102. In some examples, processing circuitry can direct subsequent care by determining a treatment plan for patient 102 based on the quality of life determination from the prediction model 118.

QOL determination system 100 includes a computing system 108 having one or more computing devices, such as a mobile computing device (e.g., a smartphone, a tablet computer, a personal digital assistant, and the like), a desktop computing device, a server system, a distributed computing system (e.g., a “cloud” computing system), and/or any other device capable of receiving patient data 104 and performing the techniques described herein.

In examples described herein, computing system 108 includes processing circuitry 126 (e.g., one or more processors) configured to execute at least one artificial intelligence (AI), deep-learning (DL), or machine-learning (ML)-based QOL predictive model or algorithm 118, in order to rapidly and accurately evaluate patient trauma. In other examples, predictive model 118 may utilize other algorithms that are trained based on groups of patient data in order to predict the QOL for other patients based on that patient-specific data. In some examples, QOL predictive model 118 may include, as non-limiting examples, logistic regression models and/or random-forest models, configured to construct a pair of predictive algorithms based on clinically relevant patient-parameter variables included in patient data 104 to predict QOL. In some such examples, restricted cubic splines may be used to model nonlinear determinative factors. System 108 or another system may develop, train, and/or evaluate the prediction model 118 using training data from a cohort of patients that have also experienced TBI. Example variables that are used in this training data are described herein. Different factors may be subject to different coefficients or weights, which may be determined by training the predictive model using patient data. In some examples, the cohort of patients may include patients that experienced a trauma, such as TBI in that example, and subsequently died. Therefore, quality of life determinations can be inclusive of a broad range of outcomes.

Once a suitable predictive model 118 is developed and evaluated, the model 118 may be implemented as software, such as a publicly available website and/or mobile application, for use as a convenient tool, e.g., to determine the predicted QOL designation 112 for patient 102 based on factors such as the patient observed outcomes for that patient. In this manner, system 108 may apply predictive model 118 to the patient-specific data as described herein.

In some examples, processing circuitry 126 may determine a treatment plan for patient 102 based on the QoL designation. This determination may include excluding one or more treatment options from a treatment plan and/or including one or more treatment options from the treatment plan. For example, if the QoL determination is low for the patient, processing circuitry 126 may select a set of one or more treatment options appropriate for that low quality of life. This may include excluding various treatment options where the potential benefit does not outweigh the potential complications or the patient is not likely to experience benefits from the treatment option. Conversely, processing circuitry 126 may expressly include one or more treatment options according to the QoL designation. For example, if the QoL designation is high, processing circuitry 126 may output more aggressive treatment options that may restore more function because the patient may be more likely to experience more benefits from that treatment. In some examples, processing circuitry 126 may determine the treatment plan as part of a triage effort to increase the utility of available medical resources to different patients.

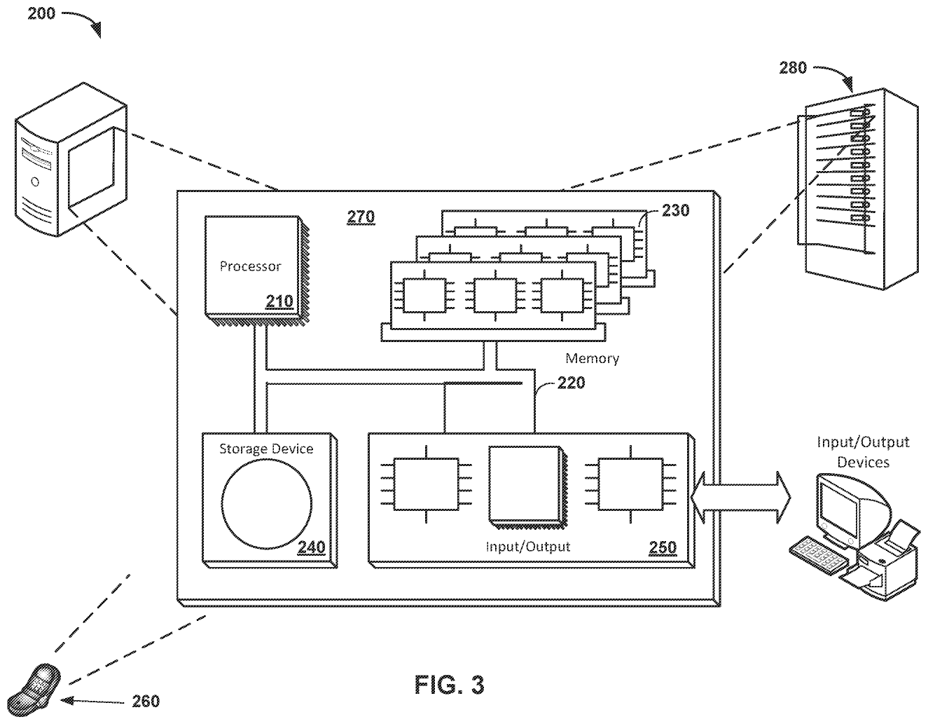

FIG. 3 is a block diagram illustrating a detailed example of various devices that may be configured to implement one or more techniques of the present disclosure. That is, computing system 200 of FIG. 2 provides an example implementation of computing system 108 of FIG. 1 for determining an QOL designation 122 for a patient 104. Computing system 200 may include one or more of a mobile device (e.g., a smartphone, laptop, tablet, a personal digital assistant [PDA], or other mobile device) 260, a workstation, a computing center, a cluster of servers 280, and/or other examples of a computing environment 200, centrally located or distributed, that is capable of executing the techniques described herein. Any or all of the devices may, for example, implement portions of the techniques described herein for determining an QOL designation (e.g., favorable or non-favorable QOL) designation 122 for trauma patients 102. In some examples, functionality of QOL determination system 100 may be distributed across multiple computing devices, such as a cloud-based computing system for computing the determined QOL designations 122 and generating corresponding reports, and a client device, such as a tablet or mobile phone, for accessing and viewing the reports.

In the example of FIG. 2, computing device 270 includes a processor 210 that is operable to execute program instructions or software, causing the computer to perform various methods or tasks, such as performing the techniques for generating and/or using multiparametric models for QOL determination as described herein. Processor 210 is coupled via bus 220 to a memory 230, which is used to store information such as program instructions and/or other data while the computer is in operation. A storage device 240, such as a hard disk drive, nonvolatile memory, or other non-transient storage device stores information such as program instructions, data files of the multidimensional data and the reduced data set, and other information. The computer also includes various input-output elements 250, including parallel or serial ports, USB, Firewire or IEEE 1394, Ethernet, and other such ports to connect the computer to external devices such a printer, video camera, display device, medical imaging device, surveillance equipment, or the like. Other input-output elements include wireless communication interfaces such as Bluetooth, Wi-Fi, and cellular data networks.

Computing device 270 may itself be a traditional personal computer, a rack-mount or business computer or server, or any other type of computerized system. Computing device 270, in a further example, may include fewer than all elements listed above, such as a thin client or mobile device having only some of the shown elements. In another example, computing device 270 is distributed among multiple computer systems, such as a distributed server that has many computers working together to provide various functions.

In one non-limiting example, computing system 200 includes a mobile computing device 260 and a remote server 280 configured to collectively execute the functionality of computing system 108 of FIG. 2. For instance, a user (e.g., clinician) may submit user data 104 via a user interface on display 106 of mobile device 260. Mobile device 260 may then transmit the user data 104 (including the plurality of patient parameters) to remote server 280. Remote server 280 is configured to execute QOL predictive model 118 to determine QOL designation 122, as described above. Remote server 280 may then transmit QOL designation 122 back to mobile device 260 for display via the GUI on display 106.

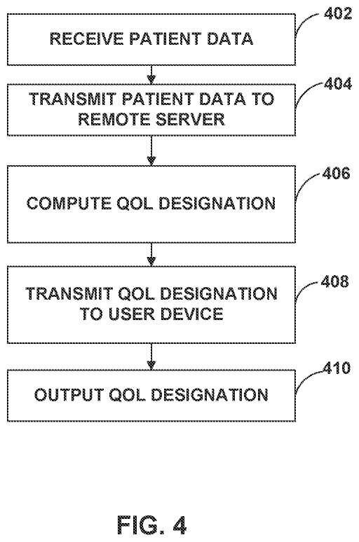

FIG. 4 is a flowchart illustrating an example operation in accordance with the techniques of this disclosure. As shown in FIG. 2, computing system 108 (e.g., a mobile computing device 260) receives initial patient parameters 104 such as medical record data and patient reported outcomes (402). In some examples, the mobile computing device 260 may transfer the patient parameters 104 to a remote server 280 of computing system 200 (404), where the patient parameters 104 may be stored into data repository 116. In this manner, remote server 280 may provide computational power for determining the QoL designation quickly and/or without needing to store predictive model 118 (and potentially sensitive data along with it) on mobile computing device 160.

Based on patient data 104, QOL predictive model 118 determines whether patient 102 is likely to have a favorable or non-favorable quality of life (406). For example, QOL predictive model 118 may be configured to determine a patient QOL designation 122 by submitting patient parameters 104 to one or more machine-learning models or predictive models or algorithms trained on historical trauma-patient data to determine a subsequent QOL designation for patient 102.

After determining an QOL designation 122 for patient 102, the remote server may transfer the QOL designation 122 back to user computing device 260 (408), which may then output the QOL designation for display via a GUI (410).

In some examples, the remote server may also, or alternatively, transfer a treatment plan back to user computing device 260. The remote server may determine the treatment plan based on the QOL designation. The treatment plan may have a complete list of one or more steps that should be used to treat the patient appropriate for the QOL designation for the patient. In other examples, the treatment plan may list one or more treatment options that should be excluded from treatment and/or one or more treatment options that should be included as at least part of the treatment of the patient.

The following examples are described herein.

Example 1: A computing system includes processing circuitry configured to: receive user input comprising a plurality of parameters indicating a condition of a trauma patient, the plurality of parameters comprising at least one patient reported outcome; apply the plurality of parameters to a predictive model trained to determine, based on the plurality of parameters, a quality of life designation for the patient; and transmit the quality of life designation to a user computing device for display on the user computing device.

Example 2: The computing system of example 1, wherein the quality of life designation comprises a favorable quality of life designation or a non-favorable quality of life designation.

Example 3: The computing system of any of examples 1 and 2, wherein the plurality of parameters comprises one or more parameters obtained from a medical record of the trauma patient.

Example 4: The computing system of any of examples 1 through 3, wherein the processing circuitry is configured to train the predictive model with respective medical record data and respective patient reported outcomes from each subject of a plurality of subjects.

Example 5: The computing system of example 4, wherein the medical record data and respective patient reported outcomes from the plurality of subjects includes patients that died from the trauma.

Example 6: The computing system of any of examples 1 through 5, wherein the processing circuitry is configured to determine, based on the quality of life determination, a treatment plan for the patient.

Example 7: The computing system of any of examples 1 through 6, wherein the processing circuitry is configured to determine the treatment plan by at least excluding one or more treatment options from the treatment plan.

Example 8: The computing system of any of examples 1 through 7, wherein the processing circuitry is configured to determine the treatment plan by at least including one or more treatment options as part of the treatment plan.

Example 9: The computing system of any of examples 1 through 8, wherein the trauma comprises traumatic brain injury.

Example 10: A method includes receiving, by processing circuitry, user input comprising a plurality of parameters indicating a condition of a trauma patient, the plurality of parameters comprising at least one patient reported outcome; applying, by the processing circuitry, the plurality of parameters to a predictive model trained to determine, based on the plurality of parameters, a quality of life designation for the patient; and transmitting, by the processing circuitry, the quality of life designation to a user computing device for display on the user computing device.

Example 11: The method of example 10, wherein the quality of life designation comprises a favorable quality of life designation or a non-favorable quality of life designation.

Example 12: The method of any of examples 10 and 11, wherein the plurality of parameters comprises one or more parameters obtained from a medical record of the trauma patient.

Example 13: The method of any of examples 10 through 12, further comprising training the predictive model with respective medical record data and respective patient reported outcomes from each subject of a plurality of subjects.

Example 14: The method of example 13, wherein the medical record data and respective patient reported outcomes from the plurality of subjects includes patients that died from the trauma.

Example 15: The method of any of examples 10 through 14, further comprising determining, based on the quality of life determination, a treatment plan for the patient.

Example 16: The method of any of examples 10 through 15, wherein determining the treatment plan comprises excluding one or more treatment options from the treatment plan.

Example 17: The method of any of examples 10 through 16, wherein determining the treatment plan comprises including one or more treatment options as part of the treatment plan.

Example 18: The method of any of examples 10 through 17, wherein the trauma comprises traumatic brain injury.

Example 19: A non-transitory computer-readable medium comprising instructions that, when executed, causes processing circuitry to receive user input comprising a plurality of parameters indicating a condition of a trauma patient, the plurality of parameters comprising at least one patient reported outcome; apply the plurality of parameters to a predictive model trained to determine, based on the plurality of parameters, a quality of life designation for the patient; and transmit the quality of life designation to a user computing device for display on the user computing device.

Example 20: The non-transitory computer-readable medium of example 19, further comprising instruction that cause processing circuitry to determine, based on the quality of life determination, a treatment plan for the patient.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media, which includes any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable storage medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

What is claimed is:

1. A computing system comprising:
processing circuitry configured to:
receive user input comprising a plurality of parameters indicating a condition of a trauma patient, the plurality of parameters comprising at least one patient reported outcome;
apply the plurality of parameters to a predictive model trained to determine, based on the plurality of parameters, a quality of life designation for the patient; and
transmit the quality of life designation to a user computing device for display on the user computing device.

2. The computing system of claim 1, wherein the quality of life designation comprises a favorable quality of life designation or a non-favorable quality of life designation.

3. The computing system of claim 1, wherein the plurality of parameters comprises one or more parameters obtained from a medical record of the trauma patient.

4. The computing system of claim 1, wherein the processing circuitry is configured to train the predictive model with respective medical record data and respective patient reported outcomes from each subject of a plurality of subjects.

5. The computing system of claim 4, wherein the medical record data and respective patient reported outcomes from the plurality of subjects includes patients that died from the trauma.

6. The computing system of claim 1, wherein the processing circuitry is configured to determine, based on the quality of life determination, a treatment plan for the patient.

7. The computing system of claim 1, wherein the processing circuitry is configured to determine the treatment plan by at least excluding one or more treatment options from the treatment plan.

8. The computing system of claim 1, wherein the processing circuitry is configured to determine the treatment plan by at least including one or more treatment options as part of the treatment plan.

9. The computing system of claim 1, wherein the trauma comprises traumatic brain injury.

10. A method comprising:
receiving, by processing circuitry, user input comprising a plurality of parameters indicating a condition of a trauma patient, the plurality of parameters comprising at least one patient reported outcome;
applying, by the processing circuitry, the plurality of parameters to a predictive model trained to determine, based on the plurality of parameters, a quality of life designation for the patient; and
transmitting, by the processing circuitry, the quality of life designation to a user computing device for display on the user computing device.

11. The method of claim 10, wherein the quality of life designation comprises a favorable quality of life designation or a non-favorable quality of life designation.

12. The method of claim 10, wherein the plurality of parameters comprises one or more parameters obtained from a medical record of the trauma patient.

13. The method of claim 10, further comprising training the predictive model with respective medical record data and respective patient reported outcomes from each subject of a plurality of subjects.

14. The method of claim 13, wherein the medical record data and respective patient reported outcomes from the plurality of subjects includes patients that died from the trauma.

15. The method of claim 10, further comprising determining, based on the quality of life determination, a treatment plan for the patient.

16. The method of claim 10, wherein determining the treatment plan comprises excluding one or more treatment options from the treatment plan.

17. The method of claim 10, wherein determining the treatment plan comprises including one or more treatment options as part of the treatment plan.

18. The method of claim 10, wherein the trauma comprises traumatic brain injury.

19. A non-transitory computer-readable medium comprising instructions that, when executed, cause processing circuitry to:
receive user input comprising a plurality of parameters indicating a condition of a trauma patient, the plurality of parameters comprising at least one patient reported outcome;
apply the plurality of parameters to a predictive model trained to determine, based on the plurality of parameters, a quality of life designation for the patient; and
transmit the quality of life designation to a user computing device for display on the user computing device.

20. The non-transitory computer-readable medium of claim 19, further comprising instruction that cause processing circuitry to determine, based on the quality of life determination, a treatment plan for the patient.
